(19) United States
(12) Patent Application Publication
(10) Pub. No.: US 2007/0239109 A1
(43) Pub. Date: Oct. 11, 2007

(54) Rotary dilator with internal threading and methods of use

(75) Inventor: Charles Joseph Dereuil, St. Paul, MN (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 11/712,703

(22) Filed: Mar. 1, 2007

Related U.S. Application Data
(60) Provisional application No. 60/780,162, filed on Mar. 8, 2006.

(51) Int. Cl. A61M 29/00

(52) U.S. Cl. 604/96.01

(57) ABSTRACT

A device and method for dilation of lumenal stenoses. The device includes a dilator with internal threads. The internal threads of the dilator provide for enhanced ability to cannulate a stenosis by engaging external threads on a wire guide that are complementary to the internal dilator threads.

### CROSS-REFERENCE TO RELATED APPLICATIONS

This application claims priority to U.S. Provisional Application Ser. No. 60/780,162, filed Mar. 8, 2006, which is incorporated herein by reference in its entirety.

### TECHNICAL FIELD

The present invention relates generally to medical devices, and more specifically to a rotary dilator device useful for dilation of stenotic lumenal occlusions, as well as methods of use for the device.

### BACKGROUND

Stenotic lumenal occlusions, whether benign or malignant, may be caused by any of a variety of ailments and may occur in any portion of the gastrointestinal tract. Dilatation of these stenoses is indicated whenever there is associated clinically significant functional impairment or a need to access beyond the stricture for diagnosis or therapy. Several different dilator devices have been used for dilation of digestive tract strictures, including those in the biliary ducts. These dilators can be delivered to strictures in a number of ways depending upon the dilator design and desired operator technique including, for example, using endoscopic, fluoroscopic, and/or wire-directed guidance. Two general classes of dilators are (1) fixed-diameter/push-type dilators and (2) expandable dilators. Each of these design classes includes “through-the-scope” designs and “non-through-the-scope” designs. “Through-the-scope” dilators are designed for use through the accessory channel of an endoscope, such as a duodenoscope. Most “non-through-the-scope” devices are deployed over a wire guide that has been placed with the aid of a subsequently-removed endoscope. Most fixed-diameter/push-type dilators are “non-through-the-scope” devices, except for some designs that are used for pancreaticobiliary applications.

Generally, dilation of a stenotic lumenal occlusion is accomplished by application of expanding forces against the lumenal stenosis. The fixed-diameter/push-type dilators exert axial as well as radial forces when they are advanced through a stenosis. These fixed-diameter/push-type dilators may be used throughout the gastrointestinal tract and can be passed therethrough via endoscopy, with or without fluoroscopy. Wire-guided through-the-scope dilators typically are passed over a wire guide and through the endoscope accessory channel. Non-through-the-scope wire-guided dilators typically are passed over a wire guide following initial placement of the wire guide using an endoscope, where the endoscope is subsequently removed prior to introduction of the dilator. Fixed-diameter/push-type dilators typically include a blunt rounded tip or an elongated tapered tip that broadens proximally. This type of dilator is typically pushed through the stenosis using a pusher-catheter, such that a smaller profile distal tip first enters the stenotic region, and then the broadening distal portion dilates the stricture as the dilator is advanced therethrough. Some stenoses are resistant to the limited amount of force that may be exerted by this type of dilator (for example, because a stenosis is too highly constricted to permit even the tip of the dilator to enter, or because the material comprising the stenosis has greater resistance than the force that can be exerted through the pusher-catheter).

Expanding dilators are typically embodied as radially-expanding balloon dilators. These balloon dilators generally are made of low-compliance materials that allow uniform and reproducible expansion to a pre-determined diameter when filled with an inflation fluid. A balloon dilator typically is advanced into a stenosed location and then expanded to dilate the stenosis. However, a balloon dilator, even when uninflated, may be too large to pass through the stenosis enough for effective deployment (by inflating the balloon).

Threaded-tip stent retrievers have also been used to dilate, for example, highly constricted pancreaticobiliary and esophageal stenotic occlusions that would otherwise allow only passage of a wire guide, and that are resistant to conventional dilation. One exemplary device is the Soehendra® stent retriever, Wilson-Cook Medical, Winston-Salem, N.C., described in U.S. Pat. Nos. 5,334,208 and 5,643,277, each of which is incorporated by reference herein. During an application for stenosis-dilation purposes, the Soehendra® stent retriever is introduced through an endoscope, over a wire-guide to a stenosed target region. The device is rotated such that the threaded exterior of its distal end augers into the stenosis, dilating it. If desired, the device may be withdrawn and another dilation device such as those described above may be used to further dilate the stenotic region.

Although such a wire-guided screw-tipped device such as a stent retriever may be used to auger through some highly constricted stenoses, other such stenoses may still prove resistant. Therefore, there is a need for a dilator system that has an improved ability to dilate resistant and/or highly constricted (such as, for example, >70% occlusion of a lumenal diameter) stenoses.

### BRIEF SUMMARY

In one aspect, the present invention provides a dilator system having an improved ability to pass through and dilate high-grade stenoses. In another aspect, the present invention further relates to methods of using the dilator system.

A dilator system embodiment of the present invention may include a dilator and a wire guide, with the dilator including an internal threaded surface adjacent its distal end. The wire guide may include a distal, externally threaded surface, with the threads being complementary to the internal threads of the dilator. The dilator may also include an external threaded surface.

In a method of the present invention, the wire guide may be used for initial cannulation of a stenotic occlusion and preferably is advanced until it engages at least a portion of the stenosis. Then, the dilator may be advanced along the wire guide until its internal threads engage the external wire guide threads. A user may then rotate one of the wire guide or the dilator relative to the other such that the dilator's internal threaded engagement with the wire guide advances the dilator distally through the stenosis. The external dilator surface, which may be threaded, may then engage the material of the stenosis and exert radial force thereupon to create a more open passage through the stenosis.

### BRIEF DESCRIPTION OF THE DRAWINGS

FIGS. 1A and 1B illustrate a first embodiment of a dilator system;

FIG. 1C illustrates a partial cross-sectional view of a wire guide of the dilator system of FIGS. 1A-1B;

FIGS. 2A and 2B depict a second embodiment of a dilator system;

FIGS. 3A and 3B depict a third embodiment of a dilator system; and

FIGS. 4A-4D show a method of using a dilator system of the present invention.

### DETAILED DESCRIPTION

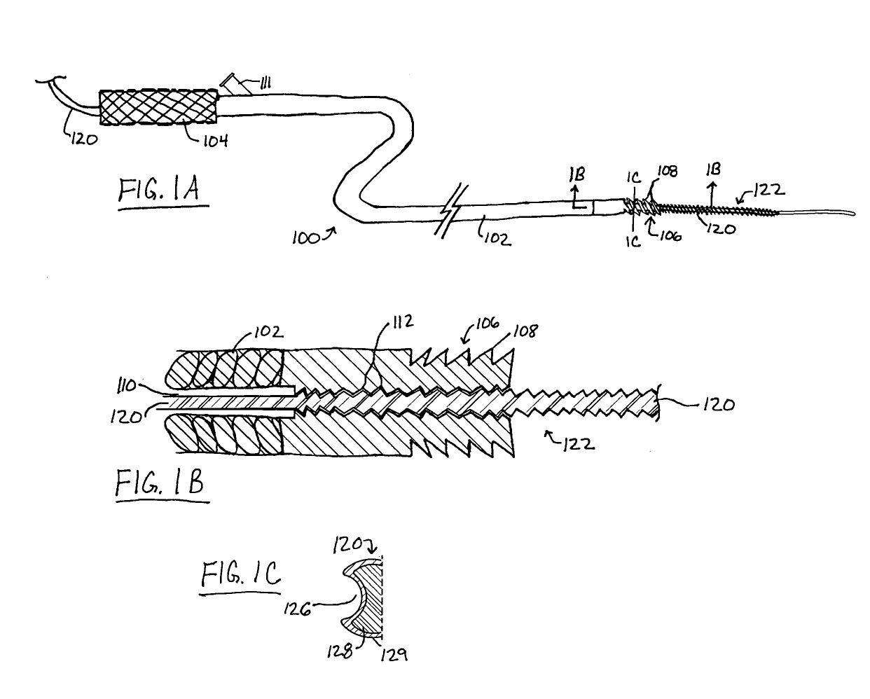

FIGS. 1A and 1B illustrate a first embodiment of a dilator 100 of the present invention. As shown in FIG. 1A, the dilator 100 includes a torqueable elongate catheter shaft 102. In the illustrated embodiment, the catheter shaft 102 includes a spiraled stainless steel wire body. A preferred shaft is flexible and efficiently transmits rotational movement from its proximal end to its distal end (i.e., torqueable). Other shaft constructions may be used with the present invention. Any shaft preferably has a lubricious surface (e.g., coated with PTFE) to ease advancement and rotation of the dilator. The proximal end includes a rotational handle 104, which has a textured surface for ease of use in gripping and rotation.

The distal end of the dilator 100 has a generally cylindrical end tip 106 that includes external helical threads 108 and preferably is less flexible than the shaft 102. The outermost diameter of the external threads 108 is substantially the same as the outer diameter of the shaft 102. The dilator 100 has a lumen 110 extending through its length. (See FIG. 1B). In the embodiment illustrated in FIGS. 1A and 1B, the dilator 100 is shown with a wire guide 120 extending through the lumen 110. The wire guide 120 has an external helically threaded portion 122, which extends along a discrete portion of the wire guide length adjacent its distal end. The outermost diameter of the wire guide threads 122 is greater than the outer diameter of the unthreaded portion of the wire guide 120.

The wire guide 120 may include an external channel 126 along at least the distal portion of its length. The channel 126 provides a path for introduction of a fluid from a fluid introduction port 111 through the lumen 110 of the dilator shaft 102, even when the external diameter of the wire guide 120 is nearly the same as the internal diameter of the lumen 110. The fluid may be, for example, a contrast fluid, a lubricant, a medicative fluid (e.g., a solution or suspension containing a medication such as an anti-inflammatory, an analgesic, or an antibiotic), a solvent material, any mixture thereof, or another desirable fluid. The channel 126 is more clearly shown in FIG. 1C, which is a partial view of a transverse cross-section taken along line 1C-1C of FIG. 1A (the partial view shows only the root portion/minor diameter of the screw-thread 122, and does not show the protruding/major diameter of the screw thread 122, nor the portion of the cylindrical end tip 106 substantially surrounding the wire guide). FIG. 1C also shows the core 128 and the coating 129 of the wire guide. The core 128 may be, for example, nitinol or stainless steel wire, and the coating 129 may be a polymer or other appropriate material (e.g., PTFE). In another embodiment, a channel may be provided along an interior surface of the lumen 110, a lumen may be provided through the wire guide with one or more openings to its outer surface, or a second lumen may be provided through the dilator shaft 102 such that a fluid (e.g., a contrast fluid or lubricant) may be directed to the distal end of the shaft 102.

The shaft 102 of this or other embodiments may include a radio-opaque material and/or may include radio-opaque markers. Such radio-opaque markers may be positioned at or near the tip and/or along the shaft such that they are useful under fluoroscopic viewing for a determination of, for example, distance of distal advancement or degree of rotation. A distal portion of the shaft 102 may include an electroconductive surface, which provides for electrocautery or electrocoagulation of a surface adjacent the shaft 102. For example, the threads 108 may comprise an electrocautery surface.

FIG. 1B is a detailed longitudinal cross-section of a portion of FIG. 1A, taken along line 1B-1B, and shows that the dilator lumen 110 includes internal helical threads 112 that complementarily engage the external wire guide threads 122. The engagement of the internal dilator threads 112 with the external wire guide threads 122 provides for rotating advancement of the dilator 100 relative to the wire guide 120. Thus, the dilator 100 and wire guide 120 provide a dilator system. The dilator 100 may be configured for introduction through an endoscope or may be configured for “non-through-the-scope” use.

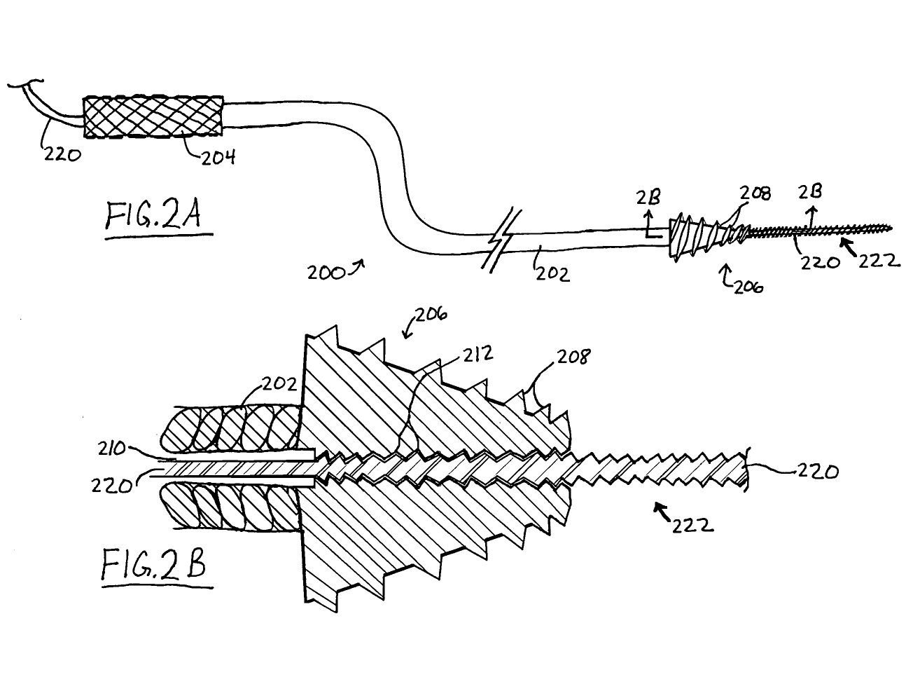

FIGS. 2A and 2B illustrate a second embodiment of a dilator 200 of the present invention. As shown in FIG. 2A, the dilator 200 has a torqueable elongate catheter shaft 202. In the illustrated embodiment, the catheter shaft 202 includes a body, which is flexible and efficiently transmits rotational movement from its proximal end to its distal end. The body may be made of multifilar tubing, for example, such as that available from Asahi-Intecc (Newport Beach, Calif.). Materials and methods of manufacturing one type of multifilar tubing are described in Published U.S. Pat. App. 2004/0116833 (Kato et al.), the contents of which are incorporated herein by reference. Other shaft constructions may be used within the present invention, and the shaft preferably has a lubricious surface (e.g., coated with PTFE) to ease advancement and rotation of the dilator. The proximal end includes a rotational handle 204, which has a textured surface for ease of use in gripping and rotation.

The distal end of the illustrated dilator embodiment 200 has a generally conical end tip 206 that includes external helical threads 208 and is preferably less flexible than the shaft 202 (the term conical as used herein is intended to encompass distal end tip shapes that would have a bullet-shaped, elliptical, or other tapered appearance in longitudinal cross-section). In the illustrated embodiment, the conical tip 206 has a base diameter greater than the outside diameter of the catheter and thereby provides for greater dilation of a stenosis than the embodiment described in FIGS. 1A-1B. It should be noted that, in certain embodiments, the angle of the conical tapering may be less than is illustrated in FIGS. 2A-2B (for example, in a different embodiment, the base diameter of a conical tip may be substantially the same as the external diameter of a catheter to which the tip is mounted). The dilator 200 has a lumen 210 extending through its length. As illustrated in FIGS. 2A and 2B, the dilator 200 is shown with a wire guide 220 extending through the lumen 210. The wire guide 220 has an external helically threaded portion 222, which extends proximally from its distal end along a discrete portion of the wire guide length. The outermost diameter of the wire guide threads 222 is greater than the outer diameter of the unthreaded portion of the wire guide 220. The threads of the dilator 200 and the wire guide 220 are shown as left-handed threads.

FIG. 2B is a detailed longitudinal cross-section of a portion of FIG. 2A, taken along line 2B-2B, and shows that the dilator lumen 210 includes internal helical threads 212 that complementarily engage the external wire guide threads 222. The engagement of the internal dilator threads 212 with the external wire guide threads 222 provide for rotating advancement of the dilator 200 relative to the wire guide 220. The dilator 200 may be configured for introduction through an endoscope or may be configured for “non-through-the-scope” use. If the dilator 200 is configured for “non-through-the-scope” use, then the conical tip 206 may include a larger base diameter than would be permitted to pass readily through the working channel of an endoscope. It should be appreciated that the thread portions of one or both of the wire guide and dilator may be single threaded or multi-threaded (such as, for example, double-threaded or triple-threaded). Embodiments with a multi-threaded portion may provide for greater advancement/retraction distances with fewer rotations of the device.

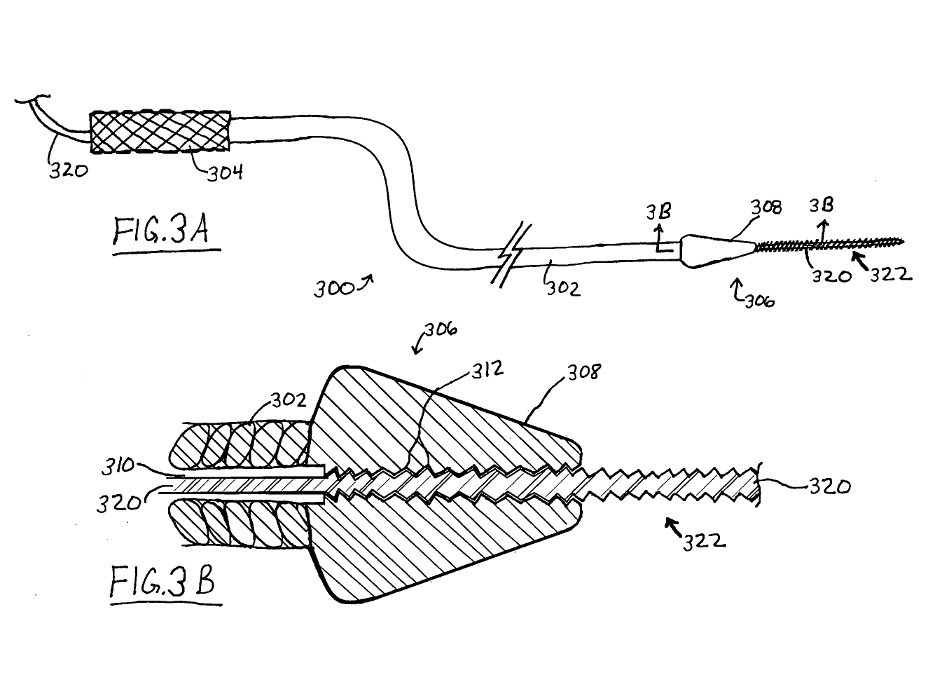

FIGS. 3A and 3B illustrate a third embodiment of a dilator 300 of the present invention. As shown in FIG. 3A, the dilator 300 has a torqueable elongate catheter shaft 302. In the illustrated embodiment, the catheter shaft 302 includes a body, which is flexible and efficiently transmits rotational movement from its proximal end to its distal end. The proximal end includes a rotational handle 304, which has a textured surface for ease of use in gripping and rotation.

The distal end of the dilator 300 has a generally conical end tip 306 that includes a generally smooth external surface 308 and preferably is less flexible than the shaft 302. Preferably, the smooth external surface 308 includes a lubricious surface coating (such as, for example, PTFE). In the illustrated embodiment, the conical tip 306 has a base diameter greater than the outside diameter of the catheter. It should be noted that, in certain embodiments, the angle of the conical tapering may be less than is illustrated in FIGS. 3A-3B (for example, in a different embodiment, the base diameter of the dilator tip may be substantially the same as the external diameter of a catheter to which the tip is mounted). The dilator 300 has a lumen 310 extending through its length. As illustrated in FIGS. 3A and 3B, the dilator 300 is shown with a wire guide 320 extending through the lumen 310. The wire guide 320 has an external helically threaded portion 322, which extends proximally from its distal end 324 along a discrete portion of the wire guide length. The outermost diameter of the wire guide threads 322 is greater than the outer diameter of the unthreaded portion of the wire guide 320.

FIG. 3B is a detailed longitudinal cross-section of a portion of FIG. 3A, taken along line 3B-3B, and shows that the dilator lumen 310 includes internal helical threads 312 that complementarily engage the external wire guide threads 322. The engagement of the internal dilator threads 312 with the external wire guide threads 322 provides for rotating advancement of the dilator 300 relative to the wire guide 320. The dilator 300 may be configured for introduction through an endoscope or may be configured for “non-through-the-scope” use. If the dilator 300 is configured for “non-through-the-scope” use, then the conical tip 306 may include a larger diameter than would be permitted to pass readily through the working channel of an endoscope. This embodiment provides a potential advantage for certain applications. Specifically, some stenoses comprise living tissue such that it may be preferable not to have an externally threaded dilator surface bitingly engaging the stenosed region. In an application using the embodiment shown in FIGS. 3A and 3B, the wire guide 320 may be advanced through the stenotic region, and then the dilator 300 may be threadedly advanced along the wire guide 320 through the stenosis, with its generally smooth surface 308 providing dilation forces that are less traumatic to surrounding material than a threaded exterior (e.g., as is illustrated in FIGS. 1A and 1B).

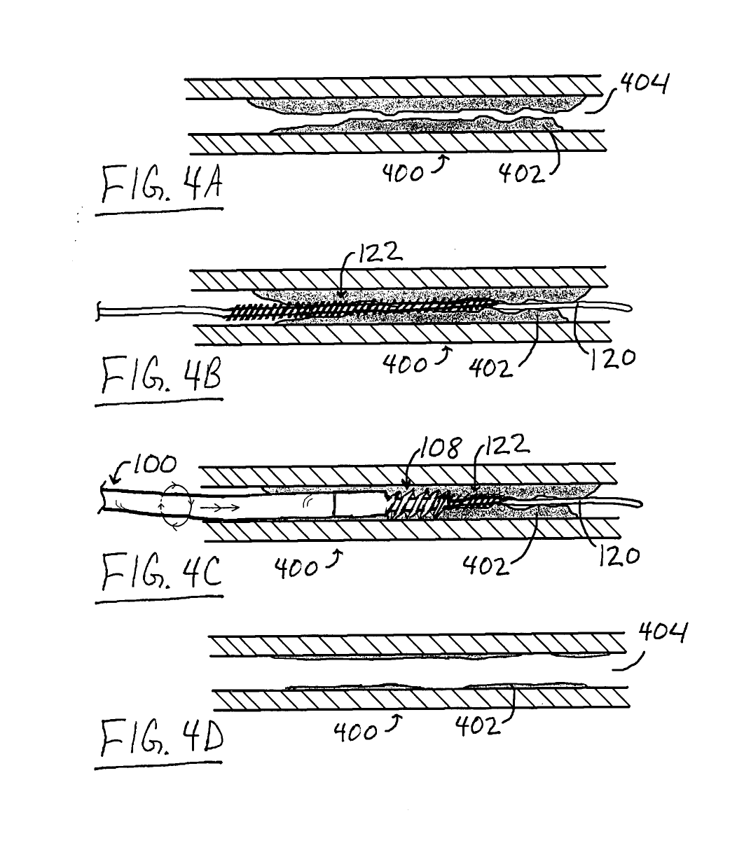

FIGS. 4A-4D illustrate a method of dilating a stenotic occlusion using the dilator system shown in FIGS. 1A-1B. FIG. 4A shows a vessel 400 with deposited material forming a stenosis 402 that significantly occludes the lumen 404 (e.g., sludge deposits in a biliary duct). As a first step of the method, shown in FIG. 4B, the wire guide 120 is introduced and passed through the stenosis 402. The threaded portion 122 of the wire guide 120 preferably traverses the stenosis 402 such that at least part of the threaded portion 122 of the wire guide 120 extends proximally from the stenosis 402. During this step, the wire guide threads 122 may help a user to rotatingly advance the wire guide 120 through a particularly tight stenosis.

Next, as depicted in FIG. 4C, the dilator 100 is advanced over the wire guide 120 to the proximal side of the stenosis 402. The user then holds the wire guide 120 in place and rotates the dilator 100 relative to the wire guide. As shown in FIG. 4C, this rotation does two things: (1) the external dilator threads 108 engage the stenosis 402 and, exerting radial force, auger through it in a manner that dilates it; and (2) to the extent the stenosis 402 is resistant to the augering movement of the dilator 100 effected by engagement of the external dilator threads 108 with the stenosis, the engagement of the internal dilator threads 112 (not shown) with the external wire guide threads 122 of the statically-held wire guide provides for axial advancement and retraction of the dilator 100 in a manner that cannulates the stenosis, allowing its dilation. After the dilator 100 is threadedly/rotatingly advanced to about the end of the wire guide threads 122, the wire guide 120 can be advancingly rotated relative to the dilator 100 to advance the wire guide 120 further through the stenosis 402. The above steps may then be repeated to dilate the next portion of the stenosis 402. FIG. 4D shows the vessel 400 after the dilator 100 has been advanced completely through the stenosis 402, after which the dilator 100 and the wire guide 120 have been withdrawn, leaving the stenosis 402 dilated such that the lumen 404 of the vessel 400 is much less occluded (such that, for example, a stent could be placed therein to aid maintenance of lumen patency). In an alternative to this method, the wire guide 120 may be held longitudinally in place and rotated relative to the dilator 100 to advance the dilator 100. In one preferred embodiment of the alternative method, the dilator 100 will not include external threads108.

It is intended that the foregoing detailed description be regarded as illustrative rather than limiting. Therefore, it is to be understood that the following claims, including all equivalents, are intended to define the spirit and scope of this invention.

I claim:

1. A dilator system, comprising:
a dilator comprising a flexible elongate catheter shaft, the catheter shaft having a proximal end, a distal end, and a lumen extending through at least a portion thereof, the shaft comprising sufficient torsional rigidity that rotational movement of the proximal end is substantially transmitted to the distal end, the lumen comprising an internal helically threaded surface extending along a portion thereof; and
a wire guide having a wire guide shaft extending between a proximal wire guide end and a distal wire guide end, the wire guide shaft comprising sufficient torsional rigidity that rotational movement of the proximal wire guide end is substantially transmitted to the distal wire guide end, the wire guide shaft further comprising an external helically threaded wire guide surface extending along a portion thereof;
wherein the external helically threaded wire guide surface is configured to engage with the internal helically threaded surface of the dilator such that a rotation of the dilator relative to the wire guide will longitudinally move the dilator relative to the wire guide.

2. The dilator system of claim 1, wherein the catheter shaft of the dilator further comprises an external helically threaded catheter shaft surface extending along a portion thereof.

3. The dilator system of claim 2, wherein the external helically threaded catheter shaft surface has a generally cylindrical shape.

4. The dilator system of claim 2, wherein the external, helically threaded catheter shaft surface has a generally conical shape.

5. The dilator system of claim 2, wherein the portion of the catheter comprising the external, helically threaded catheter shaft surface is less flexible than a major length of the catheter shaft.

6. The dilator system of claim 2, wherein the outside diameter of the external helically threaded catheter shaft surface is no greater than the outside diameter of the catheter shaft.

7. The dilator system of claim 2, wherein the external helically threaded catheter shaft surface comprises a multi-threaded surface.

8. The dilator system of claim 1, wherein each of the internal helically threaded catheter shaft surface and the external helically threaded wire guide surface comprise a multi-threaded surface.

9. The dilator system of claim 1, wherein the dilator comprises a proximal handle attached to the catheter shaft.

10. The dilator system of claim 1, wherein at least one of the catheter shaft and the wire guide further comprises radio-opaque indicia.

11. The dilator system of claim 1, wherein the wire guide further comprises a channel disposed longitudinally along its surface.

12. The dilator system of claim 1, wherein the dilator further comprises a fluid passage through at least a portion of its length.

13. A method of dilating a stenotic region in a body lumen comprising the steps of:
providing a dilator system, the dilator system comprising
a dilator comprising a flexible elongate catheter shaft, the catheter shaft having a proximal end, a distal end, and a lumen extending through at least a portion thereof, the shaft comprising sufficient torsional rigidity that rotational movement of the proximal end is substantially transmitted to the distal end, the lumen comprising an internal helically threaded surface extending along a portion thereof; and
a wire guide having wire guide shaft extending between a proximal wire guide end and a distal wire guide end, the wire guide shaft comprising sufficient torsional rigidity that rotational movement of the proximal wire guide end is substantially transmitted to the distal wire guide end, the wire guide shaft further comprising an external helically threaded wire guide surface extending along a portion thereof;
wherein the external helically threaded wire guide surface is configured to engage with the internal helically threaded surface of the dilator such that a rotation of the dilator relative to the wire guide causes longitudinal movement of the dilator relative to the wire guide;
directing the wire guide to a stenotic region in a body lumen such that at least a portion of the external helically threaded wire guide surface extends proximally adjacent the stenotic region;
advancing the dilator along the wire guide such that the internal helically threaded catheter shaft surface contacts the external helically threaded wire guide surface; and
rotating the dilator relative to the wire guide such that the internal helically threaded catheter shaft surface engages the external helically threaded wire guide surface and the dilator moves longitudinally relative to the wire guide and advances distally into the stenotic region.

14. The method of claim 13, wherein the wherein the catheter shaft of the dilator further comprises an external helically threaded catheter shaft surface extending along a distal portion thereof.

15. The method of claim 14, wherein during the rotating step the external helically threaded catheter shaft surface engages the stenotic region and aids distal advancement therethrough.

16. The method of claim 13, further comprising the step of providing a fluid through the lumen.

17. The method of claim 16, wherein the fluid is selected from the group consisting of a contrast fluid, a lubricant, a medicative fluid, a solvent, and any mixture thereof.

18. A method of dilating a stenotic region in a body lumen comprising the steps of:
providing a dilator system, the dilator system comprising
a dilator comprising a flexible elongate catheter shaft, the catheter shaft having a proximal end, a distal end, and a lumen extending through at least a portion thereof, the shaft comprising sufficient torsional rigidity that rotational movement of the proximal end is substantially transmitted to the distal end, the lumen comprising an internal helically threaded surface extending along a portion thereof; and
a wire guide having wire guide shaft extending between a proximal wire guide end and a distal wire guide end, the wire guide shaft comprising sufficient torsional rigidity that rotational movement of the proximal wire guide end is substantially transmitted to the distal wire guide end, the wire guide shaft further comprising an external helically threaded wire guide surface extending along a portion thereof;
wherein the external helically threaded wire guide surface is configured to engage with the internal helically threaded surface of the dilator such that a rotation of the dilator relative to the wire guide causes longitudinal movement of the dilator relative to the wire guide;
directing the wire guide to a stenotic region in a body lumen such that at least a portion of the external helically threaded wire guide surface extends proximally adjacent the stenotic region;
advancing the dilator along the wire guide such that the internal helically threaded catheter shaft surface contacts the external helically threaded wire guide surface; and
rotating the wire guide relative to the dilator such that the internal helically threaded catheter shaft surface engages the external helically threaded wire guide surface and the dilator moves longitudinally relative to the wire guide and advances distally into the stenotic region.

19. The method of claim 18, wherein the catheter shaft of the dilator further comprises an external helically threaded catheter shaft surface extending along a distal portion thereof.

20. The method of claim 18, wherein the distal catheter shaft end comprises a smooth surface.
